(19) United States
(12) Patent Application Publication
(10) Pub. No.: US 2003/0050227 A1
(43) Pub. Date: Mar. 13, 2003

(54) Pharmaceutical composition, reagent and method for intracerebral delivery of pharmaceutically active ingredient or labeling substance

(75) Inventor: Noriaki Kondo, Tokyo (JP)

Correspondence Address: OBLON SPIVAK MCCLELLAND MAIER & NEUSTADT PC, FOURTH FLOOR, 1755 JEFFERSON DAVIS HIGHWAY, ARLINGTON, VA, 22202, US

(73) Assignee: Mitsubishi Chemical Corporation, Tokyo (JP)

(21) Appl. No.: 10/222,857

(22) Filed: Aug. 19, 2002

Related U.S. Application Data
(63) Continuation of application No. PCT/JP01/01167, filed on Feb. 19, 2001.

(30) Foreign Application Priority Data
Feb. 18, 2000 (JP) 2000-41303

(51) Int. Cl. A61K 38/17

(52) U.S. Cl. 514/2

(57) ABSTRACT

There are provided a pharmaceutical composition which can easily transport a pharmaceutically active ingredient into a brain, a reagent can easily transport a labeling substance into a brain, and a method for delivering a pharmaceutically active ingredient or a labeling substance from blood into a brain. The pharmaceutically active ingredient and the labeling substance are bonded to a hibernation-specific protein, and used as a conjugate. By administering the conjugate together with a thyroid hormone substance or a substance enhancing secretion and production thereof, the pharmaceutically active ingredient or the labeling substance is delivered into a brain.

### TECHNICAL FIELD

The present invention relates to a pharmaceutical composition, a reagent and a method for intracerebral delivery of a pharmaceutically active ingredient or a labeling substance from blood, and so forth.

### BACKGROUND ART

The hibernation-specific protein was found as a protein of which amount changes in association with hibernation, which exists in blood of chipmunks and so forth (J. Biol. Chem., 267, 473-478 (1992)). The hibernation-specific protein includes four kinds of proteins (HP-20, HP-25, HP-27 and HP-55). HP-20, HP-25 and HP-27 have similar structures containing a collagen-like sequence (repetitions of Gly-X-Y) in about 40 residues of N-terminal amino acids, and HP-55 belongs to the α1-antitrypsin-like serpin superfamily, which shows inhibitory activity against elastase (Japanese Patent Application Laid-open (Kokai) No. 9-157299). These proteins are specifically expressed in livers of chipmunks and so forth, and secreted into blood. In blood, HP-20, HP-25 and HP-27 form a hetero trimer (HP-20c, molecular weight: about 80 kDa) by the collagen-like triple helix structure and intermolecular disulfide bonds between the half-cystine residues existing at the both termini of the triple helix structure, and HP-55 (molecular weight: about 60 kDa) is non-covalently bonded to the hetero trimer to form a complex.

In the case of chipmunks, it is known that the amount of the hibernation-specific protein circannually changes in association with hibernation, and the amount thereof in blood increases in the non-hibernating period and remarkably decreases during hibernation. To the contrary, in cerebrospinal fluid (namely, inside of a brain), it remarkably increases during hibernation period. Furthermore, the hibernation-specific protein in the cerebrospinal fluid is dissociated into HP-20c and HP-55. In the non-hibernating time with little hibernation-specific protein in cerebrospinal fluid, the hibernation-specific protein level in cerebrospinal fluid is as low as 1/1000 to 1/2000 of the level in blood. Transportation of the hibernation-specific protein into a brain is induced by administration of thyroxine (10-250 μg/kg) to animals, and the hibernation-specific protein level in cerebrospinal fluid increases within 1 day after the administration and reach a 40 to 50. times higher level within several days after the administration. Furthermore, if the administration of thyroxine is stopped, it returns to the original level.

### DISCLOSURE OF THE INVENTION

When it is intended to administer a pharmaceutically active ingredient or a labeling substance into a brain, substances that can be administered by a simple method such as administration into blood are limited, because the substance transportation from blood into a brain is strictly restricted. If there is provided a method enabling administration of a pharmaceutically active ingredient or a labeling substance into a brain by a simple method, it will become easy to transport the pharmaceutically active ingredient or the labeling substance into a brain at an organism level, and it will be extremely useful for diagnosis of brain diseases, tests for effects of drugs on the cerebral nerve system, development of novel drugs effective for the cerebral nerve system, elucidation of cerebral functions and so forth.

An object of the present invention is to provide a pharmaceutical composition which can easily transport a pharmaceutically active ingredient into a brain, a reagent which can easily transport a labeling substance into a brain, and a method for delivering a pharmaceutically active ingredient or a labeling substance from blood into a brain.

The inventor of the present invention assiduously studied in order to achieve the aforementioned object. As a result, it has been found that, if chipmunk blood plasma containing a hibernation-specific protein is intravenously administered to a nonhibernater not producing the hibernation-specific protein to which thyroxine is administered, the hibernation-specific protein level in the cerebrospinal fluid of the animal was increased, and that fragments of the proteins constituting the hibernation-specific protein complex are sufficient for the formation of the hibernation-specific protein complex. Thus, the present invention has been accomplished.

The present invention provides a pharmaceutical composition comprising a conjugate that comprises one or more pharmaceutically active ingredients bound to a hibernation-specific protein (hereinafter also referred to as “pharmaceutical composition of the present invention”), and a reagent comprising a conjugate that comprises one or more pharmaceutically active ingredients bound to a hibernation-specific protein (hereinafter also referred to as “reagent of the present invention”).

Preferably, the pharmaceutical composition of the present invention and the reagent of the present invention further comprise a thyroid hormone substance or a substance enhancing secretion and production thereof.

In the pharmaceutical composition of the present invention or the reagent of the present invention, the hibernation-specific protein may be each of the hibernation-specific proteins or a fragment thereof, or a complex constituted by them, for example, a complex between HP-20c and HP-55, a complex between a fragment of HP-20c and HP-55, a complex between HP-20c and a fragment of HP-55, a complex between a fragment of HP-20c and a fragment of HP-55 and so forth.

In the pharmaceutical composition of the present invention, the pharmaceutically active ingredient or the labeling substance may be bonded to either one or both of HP-20c or a fragment thereof and HP-55 or a fragment thereof.

The present invention also provides an agent for inducing delivery of an intracerebrally transferable protein or a conjugate that comprises one or more pharmaceutically active ingredients or one or more labeling substances bound to an intracerebrally transferable protein, into a brain, which comprises a thyroid hormone substance or a substance enhancing secretion and production thereof as an active ingredient (hereinafter also referred to as “delivery-inducing agent of the present invention”).

The present invention further provides a method for delivering a pharmaceutically active ingredient or a labeling substance into a brain, which comprises binding the pharmaceutically active ingredient or the labeling substance to a hibernation-specific protein to obtain a conjugate, and administering the conjugate together with a thyroid hormone substance or a substance enhancing secretion and production thereof (hereinafter also referred to as “delivery method of the present invention”).

### BRIEF EXPLANATION OF THE DRAWINGS

FIG. 1 shows the results of Western blot using rabbit antibodies directed to each of HP-22, HP-25, HP-27 and HP-55, which represent HP in blood and cerebrospinal fluid after administration of chipmunk plasma to thyroxine-treated rats.

FIG. 2 shows the results of immunohistochemical analysis of HP taken up into cerebral choroid epithelium cells of thyroxine-treated chipmunks.

FIG. 3 shows structures of HP-20c and HP-55. In the figure, the lines connecting two of C indicate that cystine is formed between them, and CHO indicates addition of a sugar chain. The boxed portions indicate such portions that, even if they are deleted, the association ability of HP-20c and HP-55 is maintained. The structures of HP-27, HP-25, HP-20 and HP-55 shown in the figure correspond to the sequence position of amino acid residues 31-215 shown in SEQ ID NO: 1, the sequence position of amino acid residues 29-215 shown in SEQ ID NO: 2, the sequence position of amino acid residues 24-196 shown in SEQ ID NO: 3, and the sequence position of amino acid residues 25-413 shown in SEQ ID NO: 4, respectively.

FIG. 4 shows the partial structures of HP-20c and HP-55 having the association ability. In the figure, the lines connecting two of C indicate that cystine is formed between them. SH indicates a free sulfhydryl group, and CHO indicates addition of a sugar chain. The partial structures of HP-27, HP-25, HP-20 and HP-55 shown in the figure correspond to the sequence position of amino acid residues 76-205 shown in SEQ ID NO: 1, the sequence position of amino acid residues 76-205 shown in SEQ ID NO: 2, the sequence position of amino acid residues 61-186 shown in SEQ ID NO: 3, and the sequence position of amino acid residues 279-413 shown in SEQ ID NO: 4, respectively.

### BEST MODE FOR CARRYING OUT THE INVENTION

Hereafter, embodiments of the present invention will be explained in detail.

<1> Pharmaceutical Composition of the Present Invention

The pharmaceutical composition of the present invention contains a conjugate that comprises one or more pharmaceutically active ingredients bound to a hibernation-specific protein (this will also be referred to simply as “drug conjugate” hereinafter).

In the present invention, the hibernation-specific protein (this is also abbreviated as “HP” hereinafter) means, for example, the four kinds of the hibernation-specific proteins specific for hibernation found in chipmunks, fragments thereof, or a complex constituted by those proteins or fragments thereof. The four kinds of protein are called HP-27, HP-25, HP-20 and HP-55, and their typical amino acid sequences are shown in SEQ ID NOS: 1-4, respectively. These proteins are not limited to those having the amino acid sequences shown in SEQ ID NOS: 1-4, and include allogeneic mutants thereof.

HP-27, HP-25 and HP-20 form a hetero trimer (HP-20c, molecular weight: about 80 kDa) by the collagen-like triple helix structure and intermolecular disulfide bonds between the half-cystine residues existing at the both termini of the triple helix structure, and HP-55 (molecular weight: about 60 kDa) is non-covalently bonded to the hetero trimer to form a complex. The aforementioned fragments are not particularly limited, so long as they possess an ability to form such a complex and the complex has an ability to transfer from blood into a brain, and include those fragments of the aforementioned four kinds of the proteins, obtained by partial digestion of the proteins. When HP has any activity against an animal organism to which HP is administered, to affect the animal organism, it is effective to delete or inactivate an active center of HP. The ability to form a complex and the ability to transfer from blood into a brain can be evaluated by such methods as described in the Examples mentioned hereinafter.

The HP complex may be a complex between HP-20c and HP-55, or a complex in which either one or both of HP-20c and HP-55 are fragments thereof. A fragment of HP-20c means a complex in which at least one of the proteins constituting HP-20c is digested.

The pharmaceutically active ingredient is a substance that exerts a pharmaceutical action on brain cells, and is not particularly limited so long as it can transfer from blood into a brain as the conjugate that comprises one or more pharmaceutically active ingredients bound to the HP complex, according to the present invention. The pharmaceutically active ingredient may be labeled by bonding the substance for labeling as mentioned later thereto. Examples of the action of the pharmaceutically active ingredient include treatment of diseases in a brain, and specific examples of the pharmaceutically active ingredient include antitumor (anticancer) agents, antibiotics, agents for treating autoimmune deficiency syndrome (AIDS), agents for treating neuropathic diseases such as Parkinson's disease, neuro degenerative disease, multiple sclerosis, Arzheimer's disease and depression, anodynes, migraine-treating agents, and epilepsy-treating agents.

Also, examples of the pharmaceutically active ingredient having such an action, used in the present invention, include macromolecular compounds, hydrophilic compounds, and compounds having a number of polar functional groups, which by themselves can not reach a brain from blood. Specifically, examples of the antitumor agents includes methotrexate, adriamycin, cisplatin, cyclophosphamide, etoposide, and carboplatin. Examples of the antibiotics include amphotericin B, and gentamycin. Examples of the agents for treating neuropathic diseases include neurotrophic factors such as NGF (nerve growth factor), BDNF (brain-derived neurotrophic factor), CNTF (ciliary neurotrophic factor), GDNF.(glial cell line-derived neurotrophic factor), NT-3 (neurotrophin 3), NT-4 (neurotrophin 4), NT-5 (neurotrophin 5), bFGF (basic fibroblast growth factor), aFGF (acidic fibroblast growth factor), and EFG (epithelial growth factor); nerve system-acting cytokines such as interferon α, interferon β, interferon γ, IL-1 (interleukin 1), IL-2 (interleukin 2), IL-3 (interleukin 3), IL-4 (interleukin 4), IL-5 (interleukin 5), IL-6 (interleukin 6), TNF (tumor necrosis factor), GM-CSF (granulocyte macrophage colony stimulating factor), G-CSF (granulocyte colony stimulating factor), M-CSF (macrophage colony stimulating factor), and PDGF (platelet-derived growth factor); antagonists and agonists of hormones and neurotransmitters, such as somatostatin, oxytocin, vasopressin, guaranine, VIP (vasoactive intestinal polypeptide), adorenocorticotrophic hormone, CCK (cholecystokinin), substance P, bombesin, motilin, glicentin, glucagon, glucagon-like peptide, dopamine decarboxylase, tricosanthin, and adenosine; and neurotransmitters such as dopamine, GABA (γ-aminobutyric acid), acetylcholine, serotonin, noradrenalin, and various neuropeptides. Examples of the AIDS-treating agents include nucleic acid derivatives having anti-HIV activity, such as AZT (azidethymidine), dideoxyinosine and dideoxycytosine.

Derivatives of any of the above-mentioned pharmaceutically active ingredients are also used as the pharmaceutically active ingredient in the present invention.

A conjugate that comprises one or more pharmaceutically active ingredients bound to HP can be formed by binding HP and the pharmaceutically active ingredient(s) by a method generally known as a method for binding a protein and one or more pharmaceutically active ingredients. Binding mode and binding sites of HP to the pharmaceutically active ingredient(s) are not particularly limited so long as the formed conjugate can transfer from blood into a brain. The ability to transfer from blood into a brain can be evaluated by such a method as described in the Examples mentioned hereinafter. Specifically, binding of HP and the pharmaceutically active ingredient may be performed by introducing an appropriate functional group at a site not affecting the activity of the pharmaceutically active ingredient, if necessary, and using an appropriate crosslinker and the like. Examples of the crosslinker include ones forming a peptide bond such as EDAC (1-ethyl-3-(3-dimethylaminopropyl)carbodiimide), ones forming a disulfide bond such as SPDP (N-succinimidyl-3-(2-pyridylthio)propionate) and cystamine, but are not limitted thereto. Also, protein crosslinkiers having additional function such as spacer function may be used and examples thereof include glutaraldehyde, DIPS (4,4′-diisothiocyanosthlbene-2,2′-disulfonic acid), EMCS (N-(E-maleimidocaproyloxy)succinimide), GMBS (N-(gamma-maleimidobutyloxy)succinimide), DSP (dithiobis(succinimidylpropionate) and DTBPA (4,4′-dithiobis-phenylazaide). Order of the formation of HP complex and the binding of the pharmaceutically active ingredient is not also limited. For example, it is possible to (1) bind a pharmaceutically active ingredient to a complex between HP-20c and HP-55, (2) bind a pharmaceutically active ingredient to HP-55, and then allow association of them with HP-20c, (3) bind a pharmaceutically active ingredient to HP-55, and then allow association of them with HP-20c, or the like. Furthermore, the pharmaceutically active ingredient may be bound to either one or both of HP-20c or a fragment thereof and HP-55 or a fragment thereof. As to the site to which the pharmaceutically active ingredient binds, of HP or a fragment thereof, because HP-20c and HP-55 forms a complex as described below, the site is preferable a site which does not inhibit binding of HP-20c or a fragment thereof and HP-55 or a fragment thereof and which is not exposed on a surface in the steric structure of the protein as much as possible. The site is preferably close to the binding site of HP-20c or a fragment thereof and HP-55 or a fragment thereof. Specifically, in the case of HP-20c, it is preferably C-terminal side from 71Cys in SEQ ID NO: 3, and 111Cys is particularly preferable because it is a free sulfhydryl group. In the case of HP-55, it is preferably C-terminal side from, for example, 278Met in SEQ ID NO: 4, more preferably from 379Gln to 413Gln. With respect to the ratio of the pharmaceutically active ingredient to be bound and HP or a fragment thereof, the pharmaceutically active ingredient may be bound to all of active groups of HP or a fragment thereof using a protein crosslinker or the like, or may be bound part of the active groups. If it is desired to bind the pharmaceutically active ingredient to a certain site-specific active group, it can be performed, for example, by masking the same kind of active groups at the other sites. Two or more kinds of the pharmaceutically active ingredients may be bound to one HP or a fragment thereof. The pharmaceutical composition prepared by binding as mentioned above may be evaluated for its ability to transfer into a brain according to such a method as described in the Examples mentioned hereinafter, or may be evaluated for activity of the pharmaceutically active ingredient, whether or not the conjugate itself has an active form or the like according to known methods, to screen one which is the best as the pharmaceutical composition of the present invention.

The conjugate can be contained in the pharmaceutical composition of the present invention also includes one in which both of one or more pharmaceutically active ingredients and one or more substances for labeling as mentioned later are bound to one HP or a fragment thereof, one in which one or more pharmaceutically active ingredients are bound to one of proteins constituting the HP complex or fragments thereof and one or more substances for labeling bound to another of the proteins or fragments thereof, and one which is a combination thereof.

The pharmaceutical composition of the present invention preferably further contains a thyroid hormone substance or a substance enhancing secretion and production thereof. In this specification, the thyroid hormone substance means a thyroid hormone, a substance exhibiting thyroid hormone-like action, or a structural analogue of a thyroid hormone.

The thyroid hormone is preferably thyroxine or triiodothyronine.

The substance exhibiting thyroid hormone-like action and the structural analogue of the thyroid hormone mean a substance exhibiting action equivalent to a thyroid hormone used in the present invention, and they are not particularly limited so long as they exert action of transferring a conjugate that comprises one or more pharmaceutically active ingredients bound to HP, from blood to a brain.

Examples of the substance enhancing secretion and production of the thyroid hormone substance include a substance enhancing production and secretion of a thyroid hormone in thyroid, and specific examples thereof include thyroid-stimulating hormone (TSH), thyrotropin-releasing hormone (TRH) and so forth.

The pharmaceutical composition of the present invention may contain a pharmaceutically acceptable carrier in addition to the drug conjugate and the thyroid hormone substance.

The administration method and the dose of the pharmaceutical composition of the present invention may be similar to the administration method and the dose of the drug conjugate as will be explained hereinafter concerning the delivery method of the present invention.

Dosage form and content of the drug conjugate of the pharmaceutical composition of the present invention can be suitably decided by those skilled in the art according to the administration method, the dose and so forth. If the pharmaceutical composition of the present invention contains the drug conjugate and the thyroid hormone substance or a substance enhancing secretion and production thereof, they do not necessarily need to be mixed and the pharmaceutical composition of the present invention include those wherein they are contained individually and combined.

<2> Reagent of the Present Invention

The reagent of the present invention contains a conjugate that comprises one or more labeling substances bound to a hibernation-specific protein (this will be also referred to simply as “labeled conjugate” hereinafter), and is used as an imaging agent for encephalography, for diagnosis of cerebral diseases, screening for elucidation of brain functions and so forth.

The hibernation-specific protein and the hibernation-specific protein complex may be the same as those described above for the pharmaceutical composition of the present invention.

The labeling substance is not particularly limited so long as it is a substance labeled with a substance usually used for labeling. Examples of the substance used for labeling include, for example, fluorescent substances, radioisotopes or the like, coloring matters, biotin and so forth. Antibodies and so forth can also be used.

As for the conjugate that comprises one or more labeling substances bound to the hibernation-specific protein, the production method, the order of binding and the structure of the labeled conjugate may be similar to those described above for the drug conjugate of the pharmaceutical composition of the present invention.

The reagent of the present invention preferably further contains a thyroid hormone substance or a substance enhancing secretion and production thereof. The thyroid hormone or the substance enhancing secretion and production thereof similar to one described above for the pharmaceutical composition of the present invention can be used.

The reagent of the present invention may contain a solvent usually used for injections, physiologically and pharmaceutically acceptable carrier and so forth, in addition to the labeled conjugate and the thyroid hormone substance or the substance enhancing secretion and production thereof.

The administration method and the dose of the reagent of the present invention can be suitably selected according to purposes of screening and so forth.

The dosage form, the content of the labeled conjugate and so forth of the reagent of the present invention can be suitably selected by those skilled in the art according to the administration method, the dose, the purpose. of screening and so forth. When the reagent of the present invention contains the labeled conjugate and the thyroid hormone substance or the substance enhancing secretion and production thereof, they do not necessarily need to be mixed, and the reagent of the present invention include those wherein they are contained individually and combined.

<3> Delivery-Inducing Agent of the Present Invention

The delivery-inducing agent of the present invention contains a thyroid hormone substance or a substance enhancing secretion and production thereof as an active ingredient, and induces delivery of an intracerebrally transferable protein or a conjugate that comprises one or more pharmaceutically active ingredients or one or more labeling substances bound to an intracerebrally transferable protein, into a brain.

The thyroid hormone substance or the substance enhancing secretion and production thereof, the pharmaceutically active ingredient and the labeling substance may be similar to those described above for the pharmaceutical composition of the present invention and the reagent of the present invention.

The intracerebrally transferable protein means a protein that can transfer from blood into a brain upon administration of the thyroid hormone substance or the substance enhancing secretion and production thereof. The ability to transfer from blood into a brain can be evaluated by, for example, measuring amounts of intracerebrally transferable protein in the brain or cerebrospinal fluid with or without administration of the thyroid hormone substance or the substance enhancing secretion and production thereof by using such a method as described in the Examples mentioned hereinafter.

Examples of the intracerebrally transferable protein include HP. The intracerebrally transferable protein may be a fragment so long as its transfer into a brain is not inhibited. Preparation of a conjugate that comprises one or more pharmaceutically active ingredients or one or more labeling substances bound to an intracerebrally transferable protein can be performed in the same manner as explained above for the preparation of the conjugate that comprises one or more pharmaceutically active ingredients or one or more labeling substances bound to HP or a fragment thereof as for the pharmaceutical composition of the present invention and the reagent of the present invention.

The delivery-inducing agent of the present invention may contain a physiologically and pharmaceutically acceptable carrier and so forth in addition to the thyroid hormone substance or the substance enhancing secretion and production thereof. The administration method and the dose of the delivery-inducing agent of the present invention may be similar to the administration method and the dose of the thyroid hormone substance or the substance enhancing secretion and production thereof explained hereinafter as for the delivery method of the present invention. Specifically, the dosage form, the content of the thyroid hormone substance or the substance enhancing secretion and production thereof and so forth of the delivery-inducing agent of the present invention can be suitably selected by those skilled in the art according to the administration method, the dose, the purpose of screening and so forth.

<4> Delivery Method of the Present Invention

The delivery method of the present invention is a method for delivering a pharmaceutically active ingredient or a labeling substance into a brain, which comprises binding the pharmaceutically active ingredient or the labeling substance to a hibernation-specific protein to obtain a conjugate, and administering the resultant drug or labeled conjugate together with a thyroid hormone substance or a substance enhancing secretion and production thereof.

The pharmaceutically active ingredient, the labeling substance, HP and the thyroid hormone substance or the substance enhancing secretion and production thereof may be similar to those explained for the pharmaceutical composition of the present invention and the reagent of the present invention.

The binding of the pharmaceutically active ingredient or the labeling substance to HP can be performed in the same manner as explained for the preparation of a conjugate that comprises one or more pharmaceutically active ingredients or one or more labeling substances bound to HP as for the pharmaceutical composition of the present invention and the reagent of the present invention.

In the delivery method of the present invention, the expression of “administering the drug or labeled conjugate together with a thyroid hormone substance or a substance enhancing secretion and production thereof” means that the drug or labeled conjugate and the thyroid hormone substance or the substance enhancing secretion and production thereof are administered so that there are attained levels of the both sufficient for causing transfer of the conjugate into a brain, and the administration is not limited to simultaneous administrations of the drug or labeled conjugate and the thyroid hormone substance or the substance enhancing secretion and production thereof. Therefore, either of the administration of the drug or labeled conjugate and the administration of the thyroid hormone substance or the substance enhancing secretion and production thereof may precede the other, or the both may be performed simultaneously. Preferably, the thyroid hormone substance or the substance enhancing secretion and production thereof is administered precedently.

Although the administration method of the drug or labeled conjugate is not particularly limited, it is preferable to administer the conjugate into blood. The administration into blood can be attained by, for example, intravenous injection. Dose can be suitably decided by those skilled in the art according to the body weight, age, condition of an object of administration, the kind of the pharmaceutically active ingredient or the labeling substance, the purpose of screening and so forth.

The administration method of the thyroid hormone substance or the substance enhancing secretion and production thereof also is not particularly limited so long as such a level of the thyroid hormone substance or the substance enhancing secretion and production thereof that the drug or labeled conjugate can transfer from blood into a brain. For example, it can be intravenously or intraperitoneally administered singly or intravenously as a pharmaceutical composition together with the drug conjugate. The dose of the thyroid hormone substance or the substance enhancing secretion and production thereof can be suitably decided by those skilled in the art according to the kind of the thyroid hormone substance or the substance enhancing secretion and production thereof, the body weight, age, condition of an object of administration and so forth.

The present invention is based on the following findings.

After chipmunk plasma containing HP (HP-containing plasma) was administered to a rat, a nonhibernater, that did not produce HP, HP could be detected in the blood of the animal, but HP could not be detected in the cerebrospinal fluid. After thyroxine (250 μg/kg) was administered to a rat every day for 1 week, or one day after thyroxine (100 μg/kg) was administered to a rat, increase of HP in the cerebrospinal fluid could be detected within 2-3 hours after the administration of the HP-containing plasma in the same amount as that before the thyroxine treatment.

Further, when choroid plexus epithelium cells of chipmunk in which intracerebral HP was increased by thyroxine treatment (10-250 μg/kg/day) were immunohistochemically stained by using an anti-HP-20c antibody, a strong positive reaction to the anti-HP-20c antibody was detected in the cytoplasm. This demonstrated that HP in blood was transported into the cerebrospinal fluid through choroid plexus, which is the blood-cerebrospinal fluid barrier.

The above observations means an unexpected result that HP, which is a heterogenous protein for a rat, was transported from blood into cerebrospinal fluid (into a brain) through the blood-cerebrospinal fluid barrier in the rat.

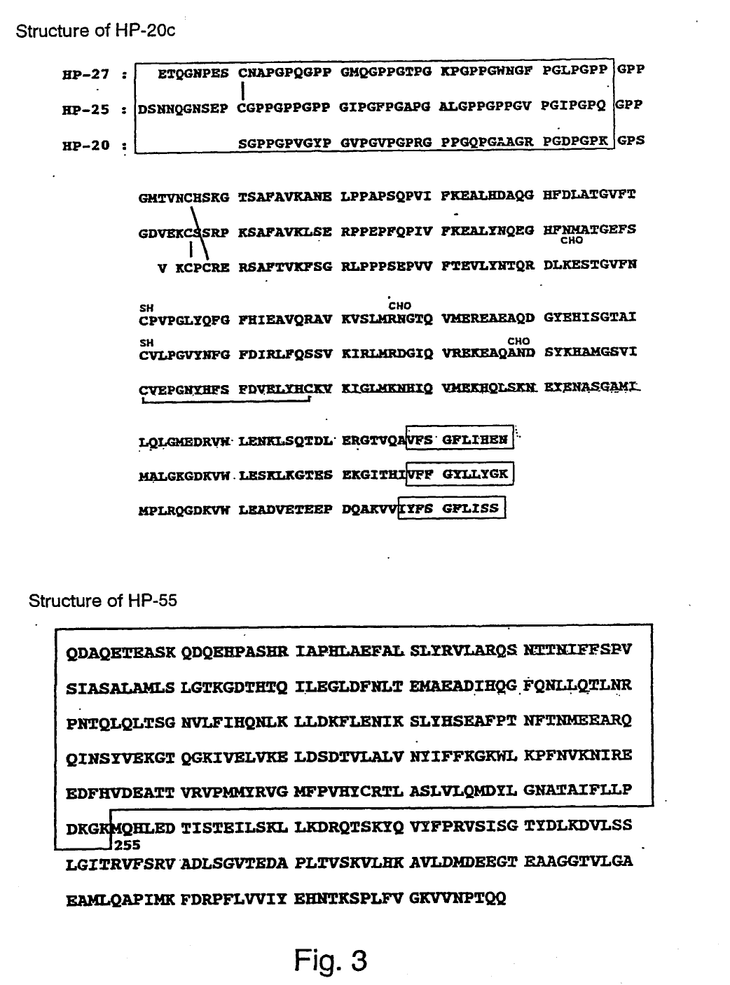

In order to determine a structure of HP effective for the transportation, structures of HP-20c and HP-55 having the ability to form a complex were investigated. As a result, HP-20c maintained the association ability with HP-55, even if about 40 of its N-terminus residues and about 10 of its C-terminus residues (boxed portions in FIG. 3) were partially deleted, and HP-55 could associate with HP-20c if about 150 residues from Met255 (this numbering corresponds to that used in the amino acid sequence shown in FIG. 3 (this residue corresponds to the amino acid number 279 in SEQ ID NO: 4)) to the C-terminus side (sequence shown in FIG. 3 except for the boxed portion) were present.

From the above results, it was found that HP-20c and HP-55 could form a complex if they had the structures mentioned above. Therefore, since it is not considered that conservation of the whole structure of HP is essential for the transportation from blood into a brain, it is concluded that HP can be modified without affecting the transfer from blood into a brain.

Based on the above results, there was established a system for transporting a pharmaceutically active ingredient or a labeling substance from blood into a brain after administrations of a conjugate that comprises one or more pharmaceutically active ingredients or one or more labeling substances bound to HP, and the thyroid hormone substance into blood.

By using the method of the present invention utilizing HP and the thyroid hormone or the substance enhancing secretion and production thereof, intracerebral administration of an arbitrary pharmaceutically active ingredient or an arbitrary labeling substance can be artificially controlled. That is, the method of the present invention makes it possible to transport an arbitrary desired pharmaceutically active ingredient or an arbitrary desired labeling substance into a brain at an arbitrary particular time by administering the pharmaceutically active ingredient or the labeling substance bound to HP into blood and utilizing activation of the transportation ability from blood into a brain caused by a thyroid hormone substance. Further, since HP forms a complex in blood and dissociates in a brain, it can also be realized that a pharmaceutically active ingredient and so forth delivered by this method should not exert its efficacy when it exists in blood, but can exert the efficacy only in a brain.

### EXAMPLE

Hereafter, the present invention will be further specifically explained with reference to the following example.

### Example 1

(1) Transportation of HP in Blood into Rat Cerebrospinal Fluid

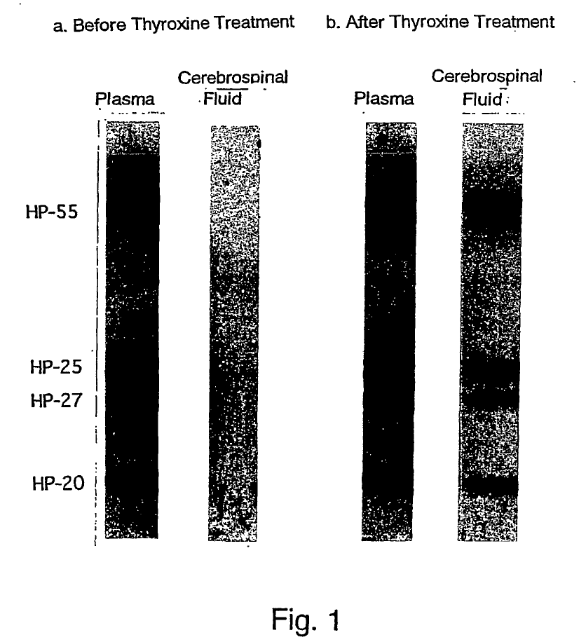

To a 12-week old rat, 0.4 ml of plasma collected from an adult chipmunk of a non-hibernating period (containing a complex between HP-20c and HP-55, referred to as “HP-containing plasma” hereinafter) was intravenously administered. Blood and cerebrospinal fluid were taken 2-3 hours after the administration, and HP in these samples was detected by Western blot using anti-HP antibodies (rabbit antibodies each directed to HP-20, HP-25, HP-27 and HP-55). The plasma in an amount of 0.13 μl and the cerebrospinal fluid in an amount of 20 μl were used for analysis. As a result, HP could be detected in the blood, but HP could not be detected in the cerebrospinal fluid (FIG. 1, a). After thyroxine was intraperitoneally administered to a rat (250 μg/kg) every day for 1 week, the HP-containing plasma was administered to the rat in the same manner as above, and HP in the blood and cerebrospinal fluid was detected. As a result, increase of HP could be detected in the cerebrospinal fluid within 2 to 3 hours after the administration. The results obtained after 2 hours are shown in FIG. 1, b. When the HP-containing plasma was administered once to a rat in the same manner as above 1 day after intraperitoneal administeration of thyroxine (100 μg/kg), the same results were obtained.

(2) Mechanism of Transportation

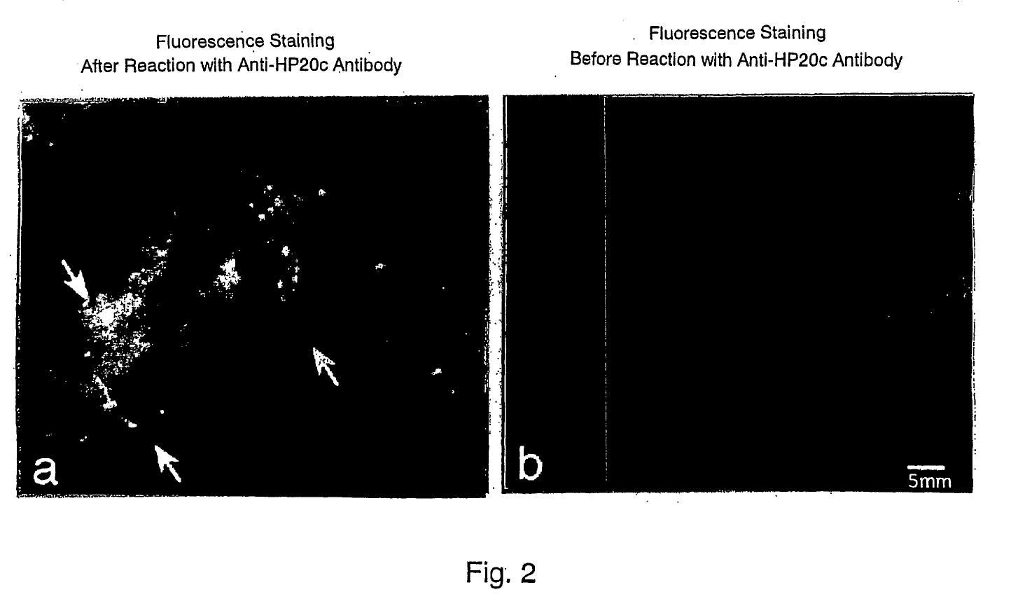

Thyroxine was intraperitoneally administered to an adult chipmunk at a non-hibernating period at a dose of 10 to 250 μg/kg/day for six days. Intracerebral choroid plexus epithelium cells were isolated from this thyroxine-treated chipmunk. This sample was immunohistochemically stained by using an anti-HP-20c antibody. As a result, a strong positive reaction to the anti-HP-20c antibody was detected in the cytoplasm (FIG. 2). This demonstrated that HP in blood was transported into the cerebrospinal fluid through choroid plexus, which is the blood-cerebrospinal fluid barrier.

(3) Determination of Modifiable Site of HP

The portions for ability to form a complex were investigated in the structures of HP-20c and HP-55. The complex between HP-20c and HP-55 dissolved in physiological saline (phosphate buffered saline, PBS) was mixed with collagenase in a weight ratio of 2:1, and incubated at 37° C. for 3 hours. The digestion products were separated by gel permeation chromatography (GPC) to isolate the partially digested HP (C-HP). C-HP in an amount of 4 mg was mixed with 1 mg of lysyl endopeptidase, and incubated at 37° C. for 5 hours for further partial digestion. The digestion products were separated by GPC to isolate the partially digested HP forming the complex. The obtained partially digested HP was further digested with lysyl endopeptidase in 50 mM Tris/hydrochloric acid buffer (pH 8.5) in the presence of 4 M urea, and the digestion products were separated by reverse phase HPLC. The peaks were each collected and attributed to peptides of HP-20c and HP-55 by comparison with peptide maps of HP-20c and HP-55 prepared similarly to determine the portions having the ability to form the complex.

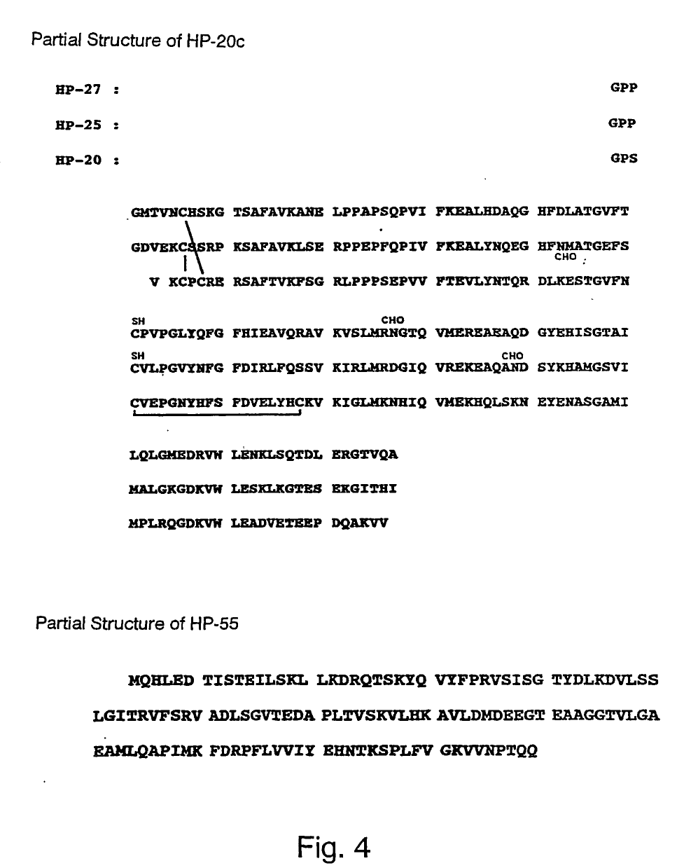

As a result, HP-20c maintained the association ability with HP-55, even if about 40 of its N-terminus residues and about 10 of its C-terminus residues (boxed portion in FIG. 3) were partially deleted, and HP-55 could associate with HP-20c if about 150 residues from Met255 (this numbering corresponds to that used in the amino acid sequence shown in FIG. 3 (this residue corresponds to the amino acid number 279 in SEQ ID NO: 4)) to the C-terminus side (sequence shown in FIG. 3 except for the boxed portion) were present. From the above results, it was found that HP-20c and HP-55 could form a complex if they had the partial structures shown in FIG. 4.

From the above results, there was established a system for transporting a pharmaceutically active ingredient or a labeling substance from blood into a brain by administrations of a conjugate that comprises one or more pharmaceutically active ingredients or one or more labeling substances bound to HP, and a thyroid hormone substance or a substance enhancing secretion and production thereof into the blood.

### INDUSTRIAL APPLICABILITY

As explained above, the present invention provides a pharmaceutical composition which can easily transport a pharmaceutically active ingredient into a brain, and a reagent which can easily transport a labeling substance into a brain. These can be effective means for treatment, prevention and diagnosis of cerebral diseases and so forth. Further, according to the present invention, a pharmaceutically active ingredient or a labeling substance can be easily delivered into a brain by administration into blood. This further makes it easy to deliver various substances into a brain at an organism level, and provides novel means for investigations including elucidation of effects of drugs on the cerebral nerve system and elucidation of cerebral functions, development of novel drugs effective for the cerebral nerve system and so forth.

1. A pharmaceutical composition comprising a conjugate that comprises one or more pharmaceutically active ingredients bound to a hibernation-specific protein.

2. The pharmaceutical composition according to claim 1, which further comprises a thyroid hormone substance or a substance enhancing secretion and production thereof.

3. The pharmaceutical composition according to claim 1 or 2, wherein the hibernation-specific protein is a complex between HP-20c and HP-55.

4. The pharmaceutical composition according to claim 1 or 2, wherein the hibernation-specific protein is a complex between a fragment of HP-20c and HP-55, a complex between HP-20c and a fragment of HP-55, or a complex between a fragment of HP-20c and a fragment of Hp-55.

5. The pharmaceutical composition according to claim 3 or 4, wherein the pharmaceutically active ingredient is bonded to either one or both of HP-20c or a fragment thereof and HP-55 or a fragment thereof.

6. A reagent comprising a conjugate that comprises one or more labeling substances bound to a hibernation-specific protein.

7. The reagent according to claim 6, which further comprises a thyroid hormone substance or a substance enhancing secretion and production thereof.

8. The reagent according to claim 6 or 7, wherein the hibernation-specific protein is a complex between HP-20c and HP-55.

9. The reagent according to claim 6 or 7, wherein the hibernation-specific protein is a complex between a fragment of HP-20c and HP-55, a complex between HP-20c and a fragment of HP-55, or a complex between a fragment of HP-20c and a fragment of HP-55.

10. The reagent according to claim 8 or 9, wherein the labeling substance is bonded to either one or both of HP-20c or a fragment thereof and HP-55 or a fragment thereof.

11. An agent for inducing delivery of an intracerebrally transferable protein or a conjugate that comprises one or more pharmaceutically active ingredients or one or more labeling substances bound to an intracerebrally transferable protein, into a brain, which comprises a thyroid hormone substance or a substance enhancing secretion and production thereof as an active ingredient.

12. A method for delivering a pharmaceutically active ingredient or a labeling substance into a brain, which comprises binding the pharmaceutically active ingredient or the labeling substance to a hibernation-specific protein to obtain a conjugate, and administering the conjugate together with a thyroid hormone substance or a substance enhancing secretion and production thereof.


SEQUENCE LISTING (4 sequences)

SEQ ID NO: 1 - protein, 215 aa, Tamias asiaticus
MYEAGKRASFMGGAGIWILALSVLMHVVCSETQGNPESCNVPGPQGPPGMRGPPGTPGKPGPPGWNGFPGLPGPPGPPGMTVNCHSKGTSAFAVKANELPPAPSQPVIFKEALHDAQGHFDLATGVFTCPVPGLYQFGFHIEAVQRAVKVSLMRNGTQVMEREAEAQDGYEHISGTAILQLGMEDRVWLENKLSQTDLERGTVQAVFSGFLIHEN

SEQ ID NO: 2 - protein, 215 aa, Tamias asiaticus
MPAQRGGALSMGAAGFWILVLSITSALADSNNQGNSEPCGPPGPPGPPGIPGFPGAPGALGPPGPPGVPGIPGPQGPPGDVEKCSSRPKSAFAVKLSERPPEPFQPIVFKEALYNQEGHFNMATGEFSCVLPGVYNFGFDIRLFQSSVKIRLMRDGIQVREKEAQANDSYKHAMGSVIMALGKGDKVWLESKLKGTESEKGITHIVFFGYLLYGK

SEQ ID NO: 3 - protein, 196 aa, Tamias asiaticus
MTDVWRLAIFVLMVNVLNDQVSCSGPPGPVGYPGVPGVPGPRGPPGQPGAAGRPGDPGPKGPSVKCPCRERSAFTVKFSGRLPPPSEPVVFTEVLYNTQRDLKESTGVFNCVEPGNYHFSFDVELYHCKVKIGLMKNHIQVMEKHQLSKNEYENASGAMIMPLRQGDKVWLEADVETEEPDQAKVVIYFSGFLISS

SEQ ID NO: 4 - protein, 413 aa, Tamias asiaticus
MPSSISWGLLLLAALSCLGPGSLAQDAQETEASKQDQEHPASHRIAPHLAEFALSLYRVLARQSNTTNIFFSPVSIASALAMLSLGTKGDTHTQILEGLDFNLTEMAEADIHQGFQNLLQTLNRPNTQLQLTSGNVLFIHQNLKLLDKFLENIKSLYHSGAFPTNFTNTEEARQQINSYVEQGTQGKIVELVKELDRDTVLALVNYIFFKGKWLKPFNVKNIREEDFHVDEATTVRVPMMYRVGMFPVHYCRTLASLVLQMDYLGNATAIFLLPDKGKMQHLEDTISTEILSKLLKDRQTSKYQVYFPRVSISGTYDLKDVLSSLGITRVFSRVADLSGVTEDAPLTVSKVLHKAVLDMDEEGTEAAGGTVLGAEAMLQAPIMKFDRPFLVVIYEHNTKSPLFVGKVVNPTQQ